(19) United States
(12) Patent Application Publication
(10) Pub. No.: US 2024/0240721 A1
(43) Pub. Date: Jul. 18, 2024

(54) Rotary Fluidic Distributor

(71) Applicant: ASP Global Manufacturing GMBH, Schaffhausen (CH)

(72) Inventors: Stuart Knowles, Oakleigh (AU); Yan Fang, Irvine, CA (US)

(73) Assignee: ASP Global Manufacturing GMBH, Schaffhausen (CH)

(21) Appl. No.: 18/289,398

(22) PCT Filed: Apr. 26, 2022
(86) PCT No.: PCT/IB2022/053876

§ 371 Date: Nov. 3, 2023

Related U.S. Application Data
(60) Provisional application No. 63/183,549, filed on May 3, 2021.

(51) Int. Cl. F16K 11/085; A61B 1/12; A61L 2/18; A61L 2/24

(52) U.S. Cl. CPC F16K 11/0856; A61L 2/18; A61L 2/24; A61B 1/125; A61L 2202/122; A61L 2202/14; A61L 2202/17; A61L 2202/24

(57) ABSTRACT

A rotary fluidic distributor suitable for outputting a pulsed flow may comprise a stator (304) defining a stator longitudinal axis and a volute, and that includes a stator conduit, and a rotor (326) comprising a rotor conduit and disposed in the volute of the stator such that a central axis of the stator conduit and a central axis of the rotor conduit are disposed in a first plane. The rotor may be rotated relative to the stator to bring the rotor conduit and the stator conduit into and out of alignment, repeatedly. The rotor conduit may comprise a plurality of rotor conduits and the stator conduit may comprise a plurality of stator conduits. The rotary fluidic distributor allows pulsed flow to be created without the use of standard valves, e.g., solenoid valves.

### FIELD

The subject matter disclosed herein relates to a technique for fluid distribution that has utility at least in automated medical-device reprocessing systems.

### BACKGROUND

Endoscopes are reusable medical devices. An endoscope should be reprocessed, i.e., decontaminated, between medical procedures in which it is used to avoid causing infection or illness in a subject. Endoscopes are difficult to decontaminate as has been documented in various news stories. See, e.g., Chad Terhune, “Superbug outbreak: UCLA will test new scope-cleaning machine,” LA Times, Jul. 22, 2015, http://www.latimes.com/business/la-fi-ucla-superbug-scope-testing-20150722-story.html. Typically, endoscope reprocessing is performed by a decontamination procedure that includes at least the following steps: removing foreign material from the endoscope, cleaning the endoscope, and decontaminating the endoscope by, among other things, submerging it in a decontaminant capable of substantially killing microorganisms thereon, e.g., infection causing bacteria.

Endoscope reprocessing may be conducted by a healthcare worker, or with the assistance of machinery, such as an endoscope reprocessor, e.g., the EVOTECH® Endoscope Cleaner and Reprocessor, manufactured by Applicant, Advanced Sterilization Products, Inc. of Irvine California.

### SUMMARY OF THE DISCLOSURE

A rotary fluidic distributor suitable for outputting a pulsed flow may comprise a stator defining a stator longitudinal axis and a volute, and that includes a stator conduit, and a rotor comprising a rotor conduit and disposed in the volute of the stator such that a central axis of the stator conduit and a central axis of the rotor conduit are disposed in a first plane. The rotor may be rotated relative to the stator to bring the rotor conduit and the stator conduit into and out of alignment, repeatedly. The rotor conduit may comprise a plurality of rotor conduits and the stator conduit may comprise a plurality of stator conduits. Additionally, the rotor conduits may be provided in sets, e.g., as a first set of rotor conduits and a second set of rotor conduits, while the stator conduits may be provided in sets, e.g., as a first set of stator conduits and a second set of rotor conduits. The first set of rotor conduits and the first set of stator conduits may be aligned with each other to provide a pulsed flow having a first pulsing frequency while the second set of rotor conduits and the second set of stator conduits may be aligned with each other to provide a pulsed flow having a second pulsing frequency. For example, there may be a greater number of rotor conduits in the first set of rotor conduits than in the second set of rotor conduits, or vice versa. The rotor may be actuated by an actuator, e.g., a stepper motor or a magnetic coupling.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements and in which:

FIG. 1 depicts an endoscope reprocessor system;

FIG. 2 depicts a schematic representation of the endoscope reprocessor system of FIG. 1;

FIG. 3 depicts an endoscope disposed in a basin of the endoscope reprocessor system of FIG. 1;

FIG. 4 depicts a schematic representation an endoscope;

FIG. 5 depicts a rotary fluidic distributor that comprises a stator and a rotor;

FIG. 6 depicts an exploded view of the rotary fluidic distributor of FIG. 5;

FIG. 7 depicts a first view of a stator body of the stator of the rotary fluidic distributor of FIG. 5;

FIG. 8 depicts a second view of the stator body of the stator of the rotary fluidic distributor of FIG. 5;

FIG. 9 depicts a cross-section view of the stator body of the stator of the rotary fluidic distributor of FIG. 5, taken along line 9-9 of FIG. 7;

FIG. 10 depicts a front view of the rotor of the rotary fluidic distributor of FIG. 5;

FIG. 11 depicts a cross-section of the rotor, taken along line 11-11 of FIG. 10;

FIG. 12 depicts a graph showing a degree of overlap between conduits of the stator and the rotor with respect to time;

FIG. 13 reflects an alternate design of a rotary fluidic distributor;

FIG. 14A reflects a simplified top cross-sectional view of a rotary fluidic distributor at a first time;

FIG. 14B reflects a simplified top cross-sectional view of a rotary fluidic distributor at a second time;

FIG. 14C reflects a simplified top cross-sectional view of a rotary fluidic distributor at a third time;

FIG. 15 reflects a simplified top cross-sectional view of a rotor having a non-radial conduit disposed therethrough;

FIG. 16 reflects the schematic of FIG. 2, modified to include a rotary fluidic distributor;

FIG. 17A depicts a graph showing pressure-decay profiles in a large-lumen endoscope channel; and

FIG. 17B depicts a graph showing pressure-decay profiles in a small-lumen endoscope channel.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

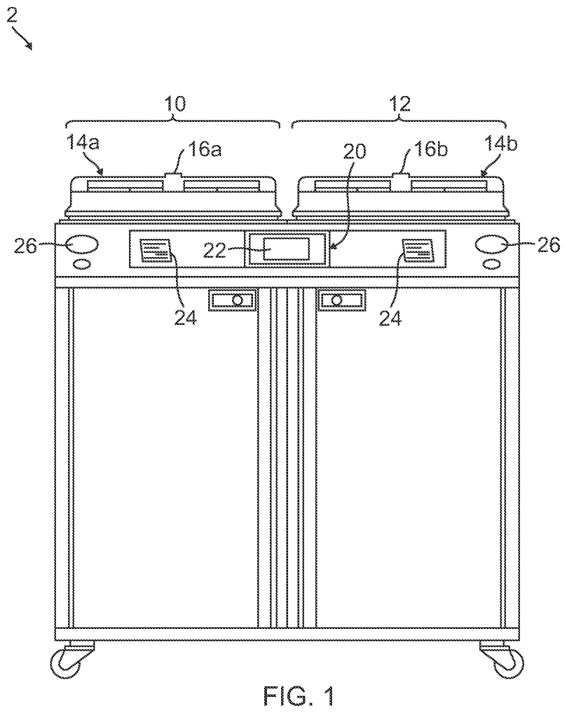

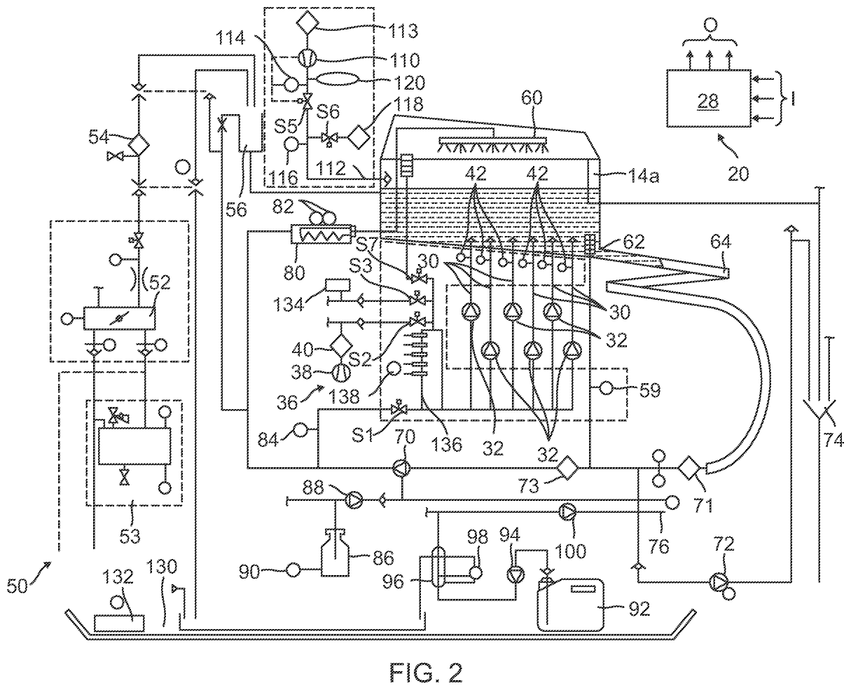

FIGS. 1-2 show an exemplary endoscope reprocessor system 2 that may be used to decontaminate (e.g., sterilize or disinfect) endoscopes and other medical devices that include channels or lumens formed therethrough. System 2 generally includes a first station 10 and a second station 12. Stations 10, 12 are at least substantially similar in all respects to provide for the decontamination of two different medical devices simultaneously or in series. First and second decontamination basins 14a, 14b receive the contaminated devices. Each basin 14a, 14b is selectively sealed by a respective lid 16a, 16b. In the present example, lids 16a, 16b cooperate with respective basins 14a, 14b to provide a microbe-blocking relationship to prevent the entrance of environmental microbes into basins 14a, 14b during decontamination operations. By way of example only, lids 16a, 16b may include a microbe removal or HEPA air filter formed therein for venting.

A control system 20 includes one or more microcontrollers, such as a programmable logic controller (PLC), for controlling decontamination and user interface operations. Although one control system 20 is shown herein as controlling both decontamination stations 10, 12, each station 10, 12 may include a dedicated control system. A visual display 22 displays decontamination parameters and machine conditions for an operator, and at least one printer 24 prints a hard copy output of the decontamination parameters for a record to be filed or attached to the decontaminated device or its storage packaging. It should be understood that printer 24 is merely optional. In some versions, visual display 22 is combined with a touch screen input device. In addition, or in the alternative, a keypad and/or other user input feature is provided for input of decontamination process parameters and for machine control. Other visual gauges 26 such as pressure meters and the like provide digital or analog output of decontamination or medical device leak testing data.

FIG. 2 diagrammatically illustrates just one decontamination station 10 of reprocessor system 2. Decontamination station 12 may be configured and operable just like decontamination station 10. It should also be understood that reprocessor system 2 may be provided with a single decontamination station 10, 12 or more than two decontamination stations 10, 12.

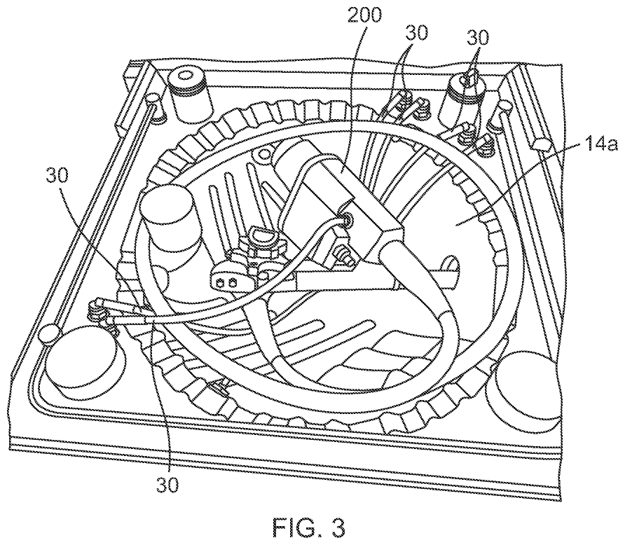

Decontamination basin 14a receives an endoscope 200 (see FIG. 3) or other medical device therein for decontamination. Any internal channels of endoscope 200 are connected with flush conduits, such as flush lines 30. Each flush line 30 is connected to an outlet of a corresponding pump 32, such that each flush line 30 has a dedicated pump 32 in this example. Pumps 32 may comprise peristaltic pumps that pump fluid, such as liquid and air, through the flush lines 30 and any internal channels of endoscope 200. Alternatively, any other suitable kind of pump(s) may be used. Pumps 32 can either draw liquid from basin 14a through a filtered drain and a valve S1; or draw decontaminated air from an air supply system 36 through a valve S2. Air supply system 36 of the present example includes a pump 38 and a microbe removal air filter 40 that filters microbes from an incoming air stream.

A pressure switch or sensor 42 is in fluid communication with each flush line 30 for sensing excessive pressure in the flush line. Any excessive pressure or lack of flow sensed may be indicative of a partial or complete blockage (e.g., by bodily tissue or dried bodily fluids) in an endoscope 200 channel to which the relevant flush line 30 is connected. The isolation of each flush line 30 relative to the other flush lines 30 allows the particular blocked channel to be easily identified and isolated, depending upon which sensor 42 senses excessive pressure or lack of flow.

Basin 14a is in fluid communication with a water source 50, such as a utility or tap water connection including hot and cold inlets, and a mixing valve 52 flowing into a break tank 56. A microbe removal filter 54, such as a 0.2 μm or smaller absolute pore size filter, decontaminates the incoming water, which is delivered into break tank 56 through the air gap to prevent backflow. A sensor 59 monitors liquid levels within basin 14a. An optional water heater 53 can be provided if an appropriate source of hot water is not available. The condition of filter 54 can be monitored by directly monitoring the flow rate of water therethrough or indirectly by monitoring the basin fill time using a float switch or the like. When the flow rate drops below a select threshold, this indicates a partially clogged filter element that requires replacement.

A basin drain 62 drains liquid from basin 14a through an enlarged helical tube 64 into which elongated portions of endoscope 200 can be inserted. Drain 62 is in fluid communication with a recirculation pump 70 and a drain pump 72. Recirculation pump 70 recirculates liquid from basin drain 62 to a spray nozzle assembly 60, described below, which sprays the liquid into basin 14a and onto endoscope 200. A coarse screen 71 and a fine screen 73 filter out particles in the recirculating fluid. Drain pump 72 pumps liquid from basin drain 62 to a utility drain 74. A level sensor 76 monitors the flow of liquid from pump 72 to utility drain 74. Pumps 70, 72 can be simultaneously operated such that liquid is sprayed into basin 14a while basin 14a is being drained, to encourage the flow of residue out of basin 14a and off of endoscope 200. Of course, a single pump and a valve assembly could replace dual pumps 70, 72.

An inline heater 80 with temperature sensors 82, upstream of recirculation pump 70, heats the liquid to optimum temperatures for cleaning and/or disinfection. A pressure switch or sensor 84 measures pressure downstream of circulation pump 70. In some variations, a flow sensor is used instead of pressure sensor 84, to measure fluid flow downstream of circulation pump 70. Detergent solution 86 is metered into the flow downstream of circulation pump 70 via a metering pump 88. A float switch 90 indicates the level of detergent 86 available. Decontaminant 92 is metered into the flow upstream of circulation pump 70 via a metering pump 94. To more accurately meter decontaminant 92, pump 94 fills a metering pre-chamber 96 under control of a fluid level switch 98 and control system 20. By way of example only, decontaminant solution 92 may comprise an activated glutaraldehyde salutation, such as CIDEX® Activated Glutaraldehyde Solution by Advanced Sterilization Products of Irvine, California. By way of further example only, decontaminant solution 92 may comprise ortho-phthalaldehyde (OPA), such as CIDEX® ortho-phthalaldehyde solution by Advanced Sterilization Products of Irvine, California. By way of further example only, decontaminant solution 92 may comprise peracetic acid (PAA).

Some endoscopes 200 include a flexible outer housing or sheath surrounding the individual tubular members and the like that form the interior channels and other parts of endoscope 200. This housing defines a closed interior space, which is isolated from patient tissues and fluids during medical procedures. It may be important that the sheath be maintained intact, without cuts or other holes that would allow contamination of the interior space beneath the sheath. Therefore, reprocessor system 2 of the present example may optionally include means for testing the integrity of such a sheath. In particular, an air pump (e.g., pump 38 or another pump 110) pressurizes the interior space defined by the sheath of endoscope 200 through a conduit 112 and a valve S5. In the present example, a HEPA or other microbe-removing filter 113 removes microbes from the pressurizing air. A pressure regulator 114 prevents accidental over pressurization of the sheath. Upon full pressurization, valve S5 is closed and a pressure sensor 116 looks for a drop in pressure in conduit 112, which would indicate the escape of air through the sheath of endoscope 200. A valve S6 selectively vents conduit 112 and the sheath of endoscope 200 through an optional filter 118 when the testing procedure is complete. An air buffer 120 smoothes out pulsation of pressure from air pump 110.

In the present example, each station 10, 12 also contains a drip basin 130 and spill sensor 132 to alert the operator to potential leaks.

An alcohol supply 134, controlled by a valve S3, can supply alcohol to channel pumps 32 after rinsing steps, to assist in removing water from channels 210, 212, 213, 214, 217, 218 of endoscope 200.

Flow rates in lines 30 can be monitored via channel pumps 32 and pressure sensors 42. If one of pressure sensors 42 detects too high a pressure, the associated pump 32 is deactivated. The flow rate of pump 32 and its activated duration time provide a reasonable indication of the flow rate in an associated line 30. These flow rates are monitored during the process to check for blockages in any of the channels of endoscope 200. Alternatively, the decay in the pressure from the time pump 32 cycles off can also be used to estimate the flow rate, with faster decay rates being associated with higher flow rates.

A more accurate measurement of flow rate in an individual channel may be desirable to detect subtler blockages. To that end, a metering tube 136 having a plurality of level indicating sensors 138 fluidly connects to the inputs of channel pumps 32. In some versions, a reference connection is provided at a low point in metering tube 136 and a plurality of sensors 138 are arranged vertically above the reference connection. By passing a current from the reference point through the fluid to sensors 138, it can be determined which sensors 138 are immersed and therefore determine the level within metering tube 136. In addition, or in the alternative, any other suitable components and techniques may be used to sense fluid levels. By shutting valve S1 and opening a vent valve S7, channel pumps 32 draw exclusively from metering tube 136. The amount of fluid being drawn can be very accurately determined based upon sensors 138. By running each channel pump 32 in isolation, the flow therethrough can be accurately determined based upon the time and the volume of fluid emptied from metering tube 136.

In addition to the input and output devices described above, all of the electrical and electromechanical devices shown are operatively connected to and controlled by control system 20. Specifically, and without limitation, switches and sensors 42, 59, 76, 84, 90, 98, 114, 116, 132 136 provide input (I) to microcontroller 28, which controls the cleaning and/or disinfection cycles and other machine operations in accordance therewith. For example, microcontroller 28 includes outputs (O) that are operatively connected to pumps 32, 38, 70, 72, 88, 94, 100, 110, valves S1, S2, S3, S5, S6, S7, and heater 80 to control these devices for effective cleaning and/or disinfection cycles and other operations.

FIG. 3 shows endoscope 200 disposed in basin 14a in a coiled configuration. As such, one portion of the endoscope's tubing overlaps or “shadows” another portion of the endoscope's tubing. The surfaces that shadow each other present a challenge to the cleaning and disinfection of the endoscope by reprocessor system 2 because the cleaning and disinfection solutions cannot readily impinge, contact, or flow over the surfaces that are overlapped by other surfaces.

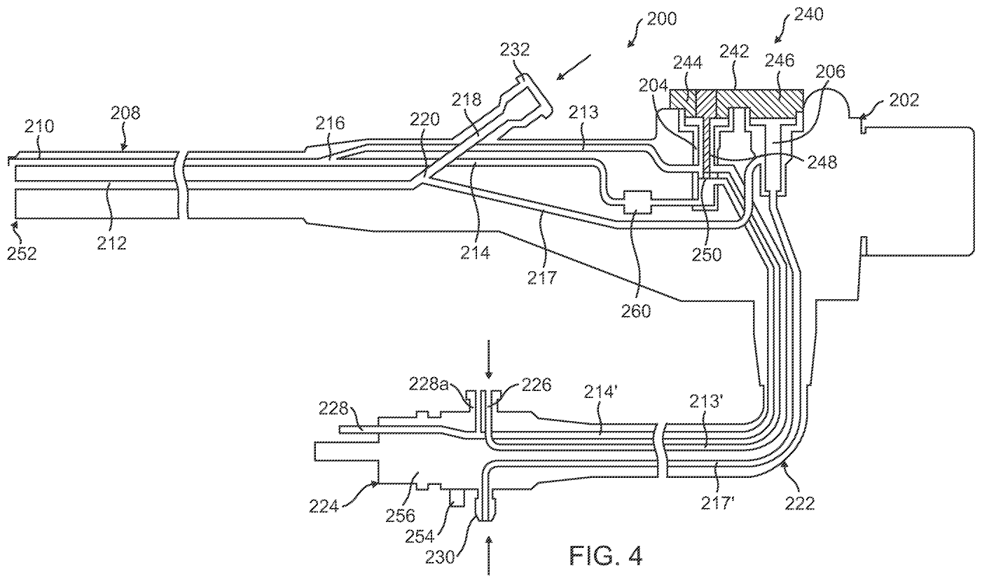

As shown in FIG. 4, endoscope 200 has a head part (control body) 202. Head part 202 includes openings 204, 206 formed therein. During normal use of endoscope 200, an air/water valve not shown and a suction valve not shown are arranged in openings 204, 206. A flexible shaft (umbilical tube) 208 is attached to head part 202. A combined air/water channel 210 and a combined suction/biopsy channel 212 are accommodated in shaft 208. A separate air channel 213 and water channel 214 are also arranged in head part 202 and merge into air/water channel 210 at the location of a joining point 216. It will be appreciated that the term “joining point” as used herein refers to an intersecting junction rather than being limited to a geometrical point and, the terms may be used interchangeably. Furthermore, a separate suction channel 217 and biopsy channel 218 are accommodated in head part 202 and merge into suction/biopsy channel 212 at the location of a joining point 220. Although endoscope 200 is shown with four independent channels (i.e., air channel 213, water channel 214, suction channel 217 and biopsy channel 218) that overlap into two combined channels (i.e., air/water channel 210 and suction/biopsy channel 212), commercially available endoscopes may have fewer channels or more channels, e.g., between one channel and eight channels. These channels may be used for different purposes. Typically, for a given endoscope, these channels each have a uniform diameter along their lengths. The lengths are dependent upon the overall length of the endoscope and whether the channel exists through the entire endoscope, such that the length of the channel equals or is substantially equal to the length of the endoscope, or only in a portion of it (e.g., between the control body and the distal end of the umbilical tube, such that the length of the channel equals or is substantially equal to the length of the umbilical tube).

An endoscope may include various channels. Diameters and lengths of these channels are provided here based on an exemplary endoscope that is approximately 3.5 meters long. It should be appreciated however, that these dimensions, particularly the lengths, may vary based on the length of the endoscope. The air channel may be used to deliver air to clear debris from the endoscope, such as a lens of the endoscope. An exemplary air channel may have a diameter of approximately 1.2 mm and comprise a first segment having a length of approximately 1700 mm and a second segment having a length of approximately 1400 mm. The suction channel may be used to aspirate fluids and debris that is directly connected thereto. An exemplary suction channel may have a diameter of approximately 1.2 mm and comprise a first segment having a length of approximately 1700 mm and a second segment having a length of approximately 1400 mm. The biopsy channel may be used to provide an entry point and passageway to the instrument channel of the endoscope. An exemplary biopsy channel may have a diameter of approximately 4.2 mm and a length of approximately 50 mm. The instrument channel may be used to provide a passageway to the distal end of the endoscope from the biopsy channel for forceps or another instrument to collect a biopsy tissue sample. An exemplary instrument channel may have a diameter of approximately 3.8 mm a length of approximately 1700 mm. Commonly, the instrument channel and biopsy channel may be collectively referred to as the biopsy channel. The water-jet channel or auxiliary-water channel may be used to deliver a jet stream of sterile fluid to wash away debris on the tissue or blood that may be blocking the view of the treatment site. An exemplary water-jet channel may have a diameter of approximately 1.0 mm and comprise a length of approximately 3500 mm. The balloon channel may be used to aspirate air fluid to fill a balloon cover that lays over the endoscope's insertion tube close to the distal tip of the endoscope to keep the field of view intact within the lumen of the GI tract. An exemplary balloon channel may have a diameter of approximately 0.8 mm and a length of approximately 2400 mm and a second segment having a length of approximately 1400 mm. Finally, the endoscope may also include an elevator channel to house a wire connected to an elevator mechanism. The wire may be manipulated to change the orientation of the elevator mechanism, which may be used to angulate forceps or other instruments at the distal tip for purposes of, e.g., endoscopic retrograde cholangiopancreatographic biopsies. An exemplary elevator channel may have a diameter of approximately 0.8 mm a length of approximately 1660 mm and a second segment having a length of approximately 1400 mm. Thus, the diameters of the channels in this exemplary endoscope vary from 0.8 mm to 4.2 mm. Further, as set forth above, some of these channels may join with others. Commonly, the water and air channels may join and the biopsy and suction channels may join.

Successful decontamination of an endoscope, such as endoscope 200, requires providing various decontaminants, e.g., decontamination liquids, such as water, detergent, disinfectant, and sterilant, or decontamination fluids (which include decontamination liquids and various gases, e.g., air or nitrogen) to all surfaces, including the inner surfaces of all of the endoscope's channels, i.e., air channel 213, water channel 214, suction channel 217, biopsy channel 218, air/water channel 210, and suction/biopsy channel 212, in sufficient volume for sufficient time. Particular challenges in endoscope reprocessing arise because endoscopes typically include between two to eight channels or lumens having small but different diameters (e.g., from approximately 0.5 millimeters to approximately 10 millimeters), which may be from approximately three meters to six meters long, and which may merge together (e.g., channels 210 and 212) or separate from each other depending on the direction of flow.

Through ongoing research and development, Applicant has determined that the efficacy of a decontamination procedure conducted on an endoscope may be improved by pulsing the flow decontamination liquids or solutions (e.g., sterilants, disinfectants, alcohol, detergent) through the endoscope's channels. As used herein, the term “pulsed flow” means varying the flow rate of a liquid or solution flowing through a single endoscope channel between a local minimum flow rate and a local maximum flow rate. The local minimum flow rate may be equal to or greater than 0 milliliters per second, and the local maximum flow rate is greater than the local minimum flow rate. As used herein, the terms “local maximum flow rate” and “local minimum flow rate” indicate that there may be variations in the pulses, e.g., the difference between the local maximum flow rate and local minimum flow rate for one pulse may be different than the difference between the local maximum flow rate and local minimum flow rate for another pulse. In other words, for a given channel, the local minimum flow rate at any given time may not be the lowest flow rate through that channel during the decontamination process, and, likewise, the local maximum flow rate at any given time may not be the greatest flow rate through that channel during the decontamination process. Pulsed flow may be contrasted with a steady flow, i.e., a flow where the flow rate of a liquid through a single endoscope channel is not varied, or at least not varied beyond concomitant variations to flow from a peristaltic pump that is pumping a liquid at a nominal flow rate.

In a reprocessor system that provides steady flow of decontamination liquids through the endoscope channels, the steady flow rate through the channels having diameters of about 0.8 mm may be between about 40 milliliters per minute and about 80 milliliters per minute, whereas the steady flow rate through the channels having diameters of about 1 mm may be between about 60 milliliters per minute and about 130 milliliters per minute, and the steady flow rate through the channels having diameters greater than about 1 mm (e.g., about 4 mm) may be between about 1000 milliliters per minute and about 2000 milliliters per minute. Where pulsed flow is to be provided, the local minimum flow rate may be less than the steady flow rate and the local maximum flow rate may be greater than the steady flow rate. Thus, for example, for channels having diameters of about 0.8 mm the local minimum flow rate may be between about 20 milliliters per minute and about 40 milliliters per minute, and the local maximum flow rate may be between about 80 milliliters per minute and about 100 milliliters per minute. For channels having diameters of about 1 mm the local minimum flow rate may be between about 30 milliliters per minute and about 60 milliliters per minute, and the local maximum flow rate may be between about 130 milliliters per minute and about 160 milliliters per minute. For channels having diameters of greater than about 1 mm (e.g., about 4 mm) the local minimum flow rate may be between about 480 milliliters per minute and 1000 milliliters per minute, and the local maximum flow rate may be between about 2000 milliliters per minute and about 2600 milliliters per minute.

The frequency with which the pulsed flow rate is pulsed, i.e., oscillates between the local minimum flow rate and the local maximum flow rate, may be between about thirty pulses per minute and about four hundred pulses per minute. The pulse frequency may be varied across the channels. For example, some channels, e.g., the channels having smaller diameters may be pulsed between about thirty pulses per minute and about ninety pulses per minute, e.g., about sixty pulses per minute, whereas other channels, e.g., the channels having larger diameters may be pulsed between about two hundred pulses per minute and about four hundred pulses per minute, e.g., three hundred pulses per minute.

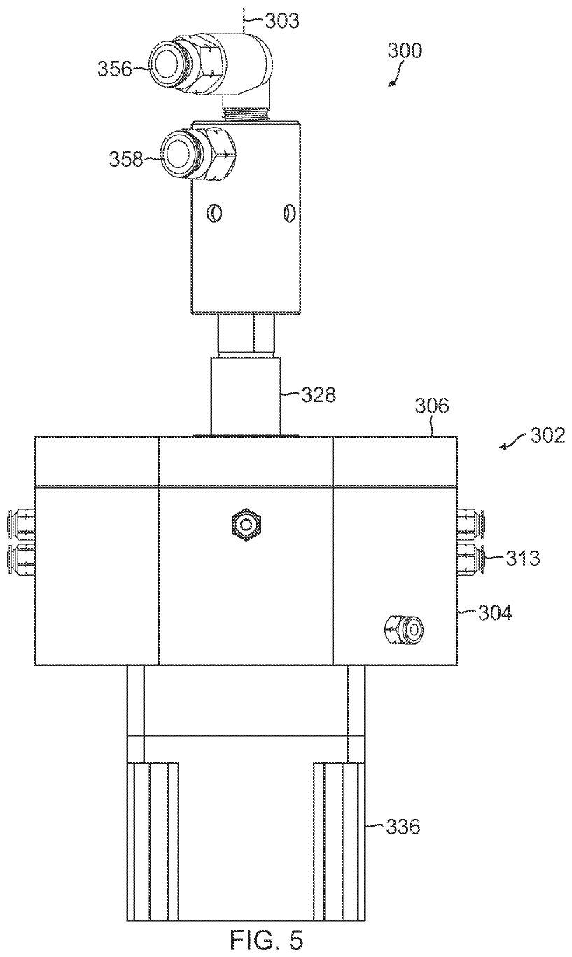

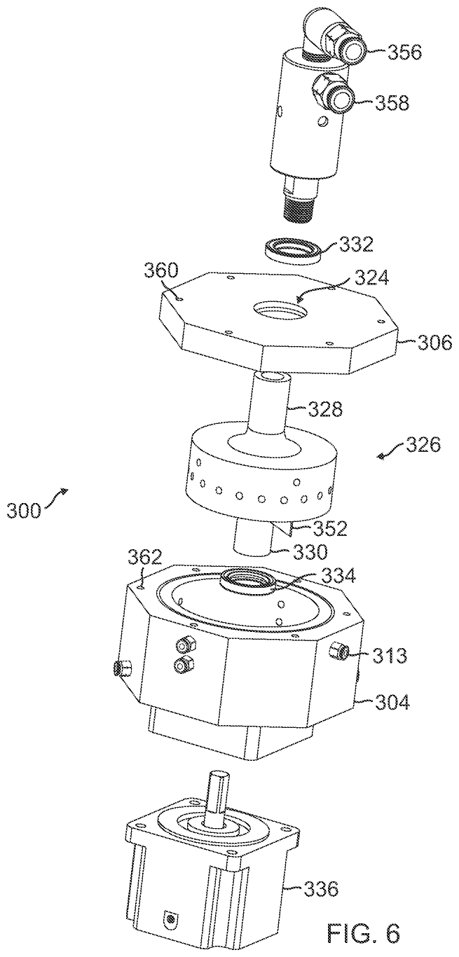

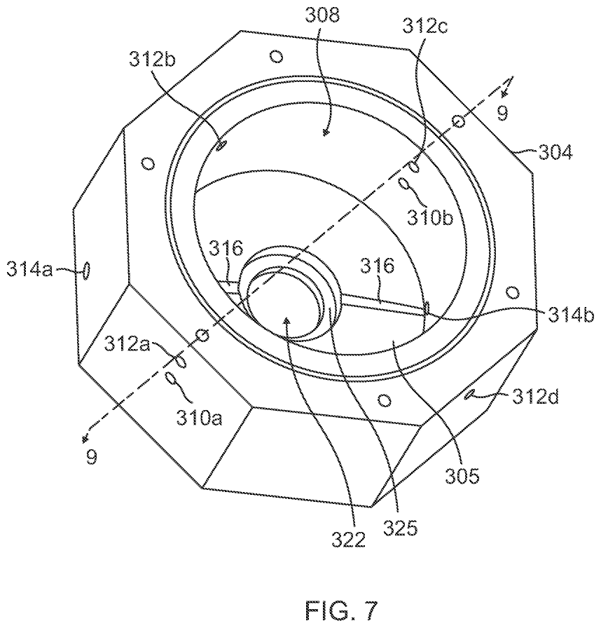

Applicant has devised a rotary fluidic distributor to assist in providing pulsed flow through channels of an endoscope being decontaminated in an endoscope reprocessor. FIGS. 5 and 6 reflect a rotary fluidic distributor 300 comprising a stator 302. Stator 302 may define a longitudinal axis 303 and be comprised of a body 304, further detailed in FIGS. 7-9, having a base 305 and a cap 306 that mates to body 304. A first hole or stator-body hole 322 may be provided through base 305, and a second hole or stator-cap hole 324 may be provided through cap 306. Body 304 may define a cavity 308 such that, when cap 306 is mated to body 304 to border cavity 308, cavity 308 may be considered a volute 308. Cap 306 may be mated to body 304 in any suitable manner. For example, as reflected in the figures, holes 360 and 362 are provided for screws or bolts.

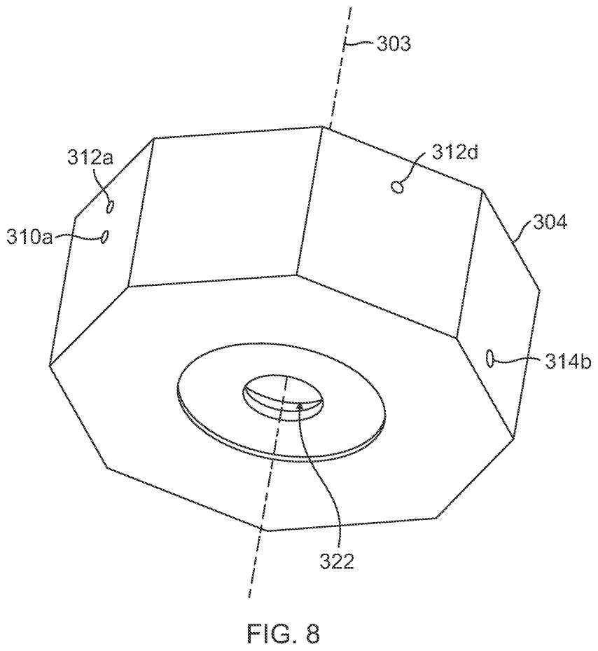

At least one conduit 310 may be disposed through stator 302. As best reflected in FIGS. 7-9, various stator conduits are provided through stator body 304, including stator conduits 310a-b, 312a-d, and 314a-b. Tube or hose connectors 313 may be mated to any one or more of these stator conduits to mate with tubing, such as flush lines 30, for transporting fluids away form rotary fluidic distributor 300, as explained below. Stator conduits 310a-b, which may be considered a first set of stator conduits, are disposed through stator body 304 at the same distance from base 305. Stator conduits 312a-d, which may be considered a second set of stator conduits, are each disposed about the stator at the same distance from base 305 of stator body 304 and above the level of stator conduits 310a and 310b. Stator conduits 314a-b, which may be considered a drainage set of stator conduits, are disposed through the stator, with a portion of their cross section just beneath base 305 and another portion of their cross section just above base 305. Drainage channels 316 in base 305 connect to stator conduits 314a-b. Each of stator conduits 310a-b, 312a-d, and 314a-b define their own central (longitudinal) axis, such as, for example, central axes 318 and 320 shown in FIG. 8 as corresponding respectively to stator conduits 310a and 312a. These central axes may be disposed transversely or perpendicularly to stator longitudinal axis 303 such that they are disposed, respectively, in a first plane and a second plane that are also perpendicular to stator longitudinal axis 303. As such, the central axis of stator conduit 310b is also in the first plane, whereas the central axes of stator conduits 312b, 312c, and 312d are also in the second plane. Furthermore, as axes 318 and 320 are offset from each other and may be parallel to each other, the first plane and second plane may also be offset from each other and parallel to each other. Although the figures and discussion herein reflect stator conduits 310a-b as being below stator conduits 312a-d, they could alternatively be above stator conduits 312a-d.

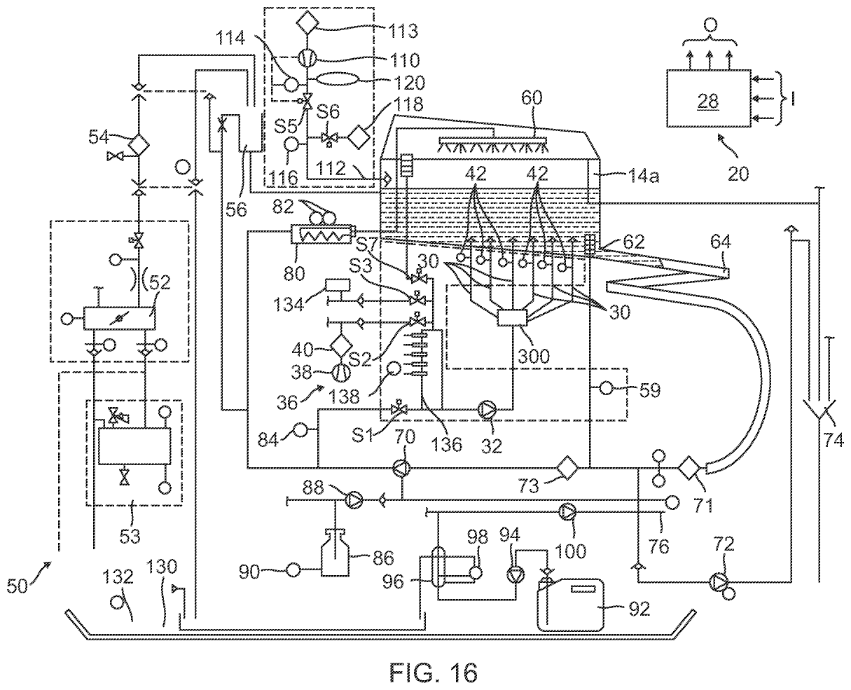

Furthermore, a greater or lesser number of stator conduits may be provided. For example, the first set of stator conduits may comprise only a single stator conduit, or it may comprise three to ten stator conduits, or even more stator conduits. Similarly, the second set of stator conduits may comprise only a single stator conduit, or it may comprise between two and ten stator conduits, or even more stator conduits. The total number of stator conduits in each set and the total number of stator conduits overall depends on the purpose of the system in which rotary fluidic distributor 300 is incorporated. For example, when incorporated into an endoscope reprocessor, such as endoscope reprocessor 2, the overall number of stator conduits should correspond to the number of channels of an endoscope, with either the first set or second set corresponding to the number of channels having larger diameters and the other of the first set and second set corresponding to the number of channels having smaller diameters. As reflected in FIGS. 3 and 16, there are six flush lines 30, indicating that reprocessor 2 is designed to be used to decontaminate and endoscope having six channels.

Rotary fluidic distributor 300 also comprises a rotor 326, depicted in FIGS. 6, 10, and 11. At least a portion 346 of rotor 326 may be disposed in cavity or volute 308 of stator 302 such that a longitudinal axis 327 of rotor 326 is coaxial with longitudinal axis 303 of stator 302. Rotor 326 also includes a first shaft 328 and a second shaft 330 that extend outwardly from portion 346 of rotor 326. Portion 346 of rotor 326 may be considered a central portion of rotor 326 inasmuch as first shaft 328 and second shaft 330 extend outwardly from portion 346. First shaft 328 may be disposed through stator-cap hole 324 and second shaft 330 may be disposed through stator body hole 322. First shaft 328 may include a first bore 338 and second shaft 330 may include a second bore 340. Fluid inputs 356 and 358 may be connected to first bore 338 of first shaft 328 for introduction of liquids and gases into rotor 326.

A seal or gasket 332 may be disposed about first shaft 328 and against stator cap 306 to minimize or prevent any leakage of fluids from inside volute 308 through any spacing between first shaft 328 and stator cap 306. Similarly, a seal or gasket 334 may be disposed about second shaft 330 and against stator body 304 to minimize or prevent any leakage of fluids from inside volute 308 through any spacing between second shaft 330 and stator body 304. Seats for seals 332 and 334 may be provided in stator 302. For example, as best seen in FIG. 7, a seat 325 for seal 334 is shown in base 305 of stator body 304.

Rotor 326 may additionally comprise various rotor conduits, including rotor conduits 332a-t and 334a-d, which may be disposed through portion 346 of rotor 326, both of which may have cylindrical forms. Rotor conduits 332a-t may be considered a first set of rotor conduits and rotor conduits 334a-d may be considered a second set of rotor conduits. These rotor conduits may be disposed through a portion 346 of rotor 326. When rotor 326 is disposed in volute 308, central axes of each of rotor conduits 332a-t are disposed in the first plane while central axes of each of rotor conduits 334a-d are disposed in the second plane. In this manner, rotor 326 may be rotated to various orientations relative to stator 302 where at least one of the rotor conduits of the first set of rotor conduits (i.e., rotor conduits 332a-t) aligns with a corresponding one or more of the stator conduits of the first set of stator conduits (i.e., stator conduits 310a-b), or at least one of the rotor conduits of the second set of rotor conduits (i.e., rotor conduits 334a-d) aligns with a corresponding one or more of the stator conduits of the second set of stator conduits (i.e., stator conduits 312a-d), or where none of the rotor conduits align with any of the stator conduits. Thus, when rotor 326 is rotated continuously about axis 327 relative to stator 302, the first set of rotor conduits rotates relative to the first set of stator conduits, bringing these conduits repeatedly into and out of alignment. Similarly, when rotor 326 is rotated continuously about axis 327 relative to stator 302, the second set of rotor conduits rotates relative second set of stator conduits, bringing these conduits repeatedly into and out of alignment. Although the figures and discussion herein reflect rotor conduits 332a-t as being below stator conduits 334a-d, they could alternatively be above rotor conduits 334a-d, particularly if there is a set of stator conduits for them to align with.

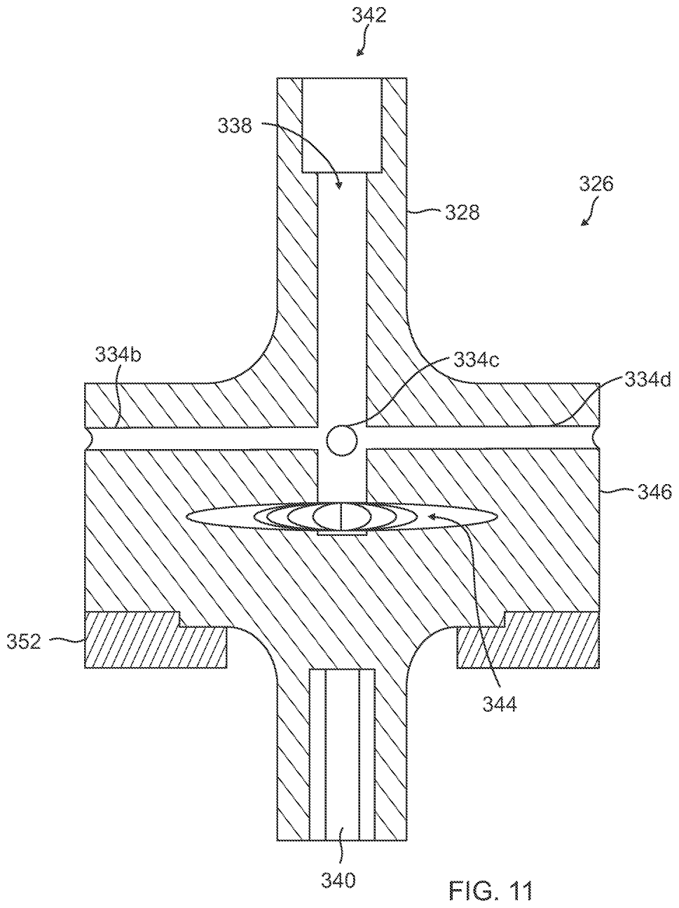

As seen in FIG. 11, a decontaminant (e.g., a decontamination fluid, such as a decontamination liquid or a gas) flowed into bore 338 through first-bore ingress 342 via inlet 356 or 358 will flow into each of the rotor conduits through a corresponding rotor-conduit ingress. Accordingly, the first bore may be connected to a source of a decontaminant. Where the source of the decontaminant comprises a decontamination liquid, a pump may also be connected between the source and the bore. Where the source of the decontaminant comprises a gas, the source may comprise a pressurized gas. The volume in FIG. 11 identified by reference numeral 344 is formed by all of the rotor conduits 332a-t overlapping each as they enter a volume defined by bore 338. Volume 344 may be considered a single rotor-conduit ingress that serves all rotor conduits 332a-t. In further embodiments more than one bore may be provided in the first or second shaft. For example, a first bore may be used to introduce liquids and a second bore may be used to introduce gases.

As a rotor conduit moves into alignment with that stator conduit, the flow rate in that stator conduit increases to a local maximum flow rate. Then, as the rotor conduit moves out of alignment with that stator conduit, the flow rate returns to a local minimum flow rate. This process repeats every time a rotor conduit moves into and then out of alignment with a stator conduit. Thus, the number of pulses per unit time in a given stator conduit depends on the number of rotor conduits that may be aligned with that stator conduit and the angular speed of the rotor.

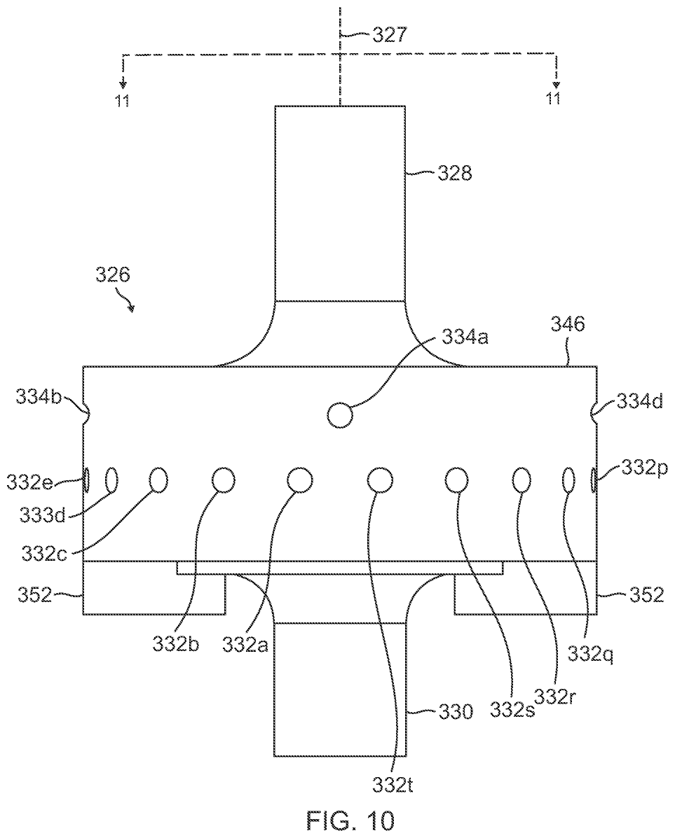

Furthermore, a greater or lesser number of rotor conduits may be provided than those reflected in FIGS. 10 and 11. For example, the first set of rotor conduits may comprise only a single stator conduit, or it may comprise three to twenty-five rotor conduits, or fifteen to twenty five rotor conduits even more rotor conduits. Similarly, the second set of rotor conduits may comprise only a single stator conduit, or it may comprise between three and five rotor conduits, or three and ten rotor conduits, even more rotor conduits. The total number of rotor conduits in each set and the total number of rotor conduits overall depends on the rate of pulsing that may be required of the flow.

Additionally, the stator conduits in the first set of stator conduits may be equally spaced from each other, or at least one may be not equally spaced from the others. Similarly, the stator conduits in the second set of stator conduits may be equally spaced from each other, or at least one may be not equally spaced from the others. Similarly, the rotor conduits in the first set of rotor conduits may be equally spaced from each other, or at least one may be not equally spaced from the others. Similarly, the rotor conduits in the second set of rotor conduits may be equally spaced from each other, or at least one may be not equally spaced from the others. Additionally, any one of the rotor conduits in the first set of rotor conduits may be horizontally displaced relative to every conduit in the second set of conduits or vice versa. For example, as seen in FIG. 10, rotor conduit 334a is disposed above but between rotor conduits 332a and 332t. Having conduits in any given set of conduits being equally spaced or not equally spaced, and whether any rotor conduits in one set should be horizontally offset from all of the other conduits in another set of rotor conduits enables flow to be pulsed to a local maximum flow rate in a given stator conduit (and corresponding endoscope channel) while having a local minimum flow rate in another stator conduit (and corresponding endoscope channel), or to have a maximum flow rate in multiple or all stator conduits (and corresponding endoscope channels), or to have a minimum flow rate in multiple or all stator conduits (and corresponding endoscope channels).

One advantage of having a local maximum flow rate in at least one stator conduit while having a flow rate of less than the local maximum flow rate, e.g., a local minimum flow rate, in all of the remaining stator conduits is that this provides an avenue for avoiding stagnation of flow or reverse flow through a channel of an endoscope. For example, with reference to FIG. 4, simultaneous flow of fluid through channels 218 and 217, ideally results in all of that fluid flowing into channel 212 and out of tip 252 of endoscope 200. However, there are difficulties in achieving this result because channel 218 provides less resistance to flow than channel 217. That is, channel 218 a larger diameter than and is shorter than channel 217. As such, it has been observed that under certain conditions, attempting simultaneous flow into channels 218 and 217 via ingresses 232 and 230 may result in stagnation somewhere in channel 217, or even backward flow therethrough. Rotary distributor 300 solves this issue because the pulses of the pulsed flow through channels 217 and 218 may be provided out of phase with each other. That is, in repeated alternation, a local maximum flow rate may be provided through channel 217 while a local minimum flow rate is being provided through channel 218, and then a local maximum flow rate may be provided through channel 218 while a local minimum flow rate is being provided through channel 217. Such is accomplished by having the stator conduit connected to channel 218 never being aligned with a rotor conduit at the same time that the stator conduit connected to channel 217 is aligned with a rotor conduit, and vice versa.

In certain applications, it may be desirable to remove liquids from the rotary fluidic distributor, particularly spent decontamination liquids at the end of a decontamination procedure. Accordingly, drainage channels and drainage conduits may be provided, as seen in FIG. 7 at reference numerals 314a-b and 316. To assist liquids to reach the drainage channels and drainage conduits, a sweep may be provided on the rotor. For example, as seen in FIG. 10, one or more sweeps 352 are provided on portion 346 of rotor 326. Inside volute 308, the bottoms of sweeps 352 contact base 305 such that rotation of rotor 326 causes them to sweep base 305. The sweep may be comprised of a synthetic rubber, e.g., neoprene.

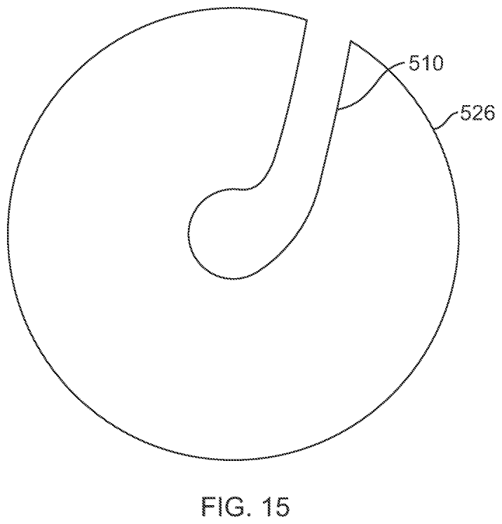

The rotor conduits may be disposed radially or non-radially about rotor longitudinal axis 327. When at least one rotor conduit is disposed non-radially as shown for rotor conduit 510 of rotor 526 in FIG. 15, flow through this rotor conduit may provide a thrust sufficient to cause rotor 526 to rotate about its longitudinal axis. For additional control, especially where all the rotor conduits in at least one set are radially disposed, an actuator should be connected to rotor 326. For example, as shown in FIG. 1, a stepper motor 336 is connected to rotor 326 via second bore 340 of second shaft 330.

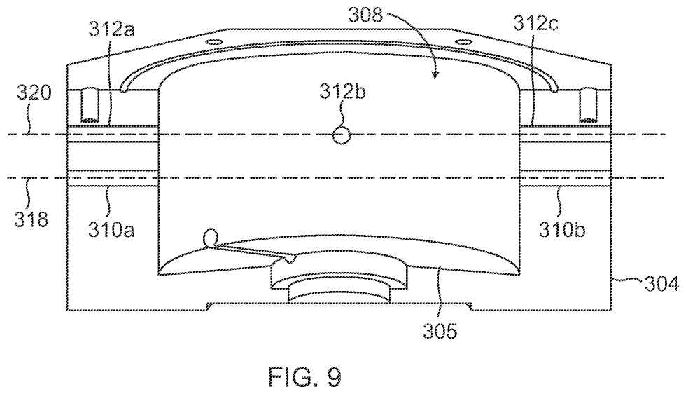

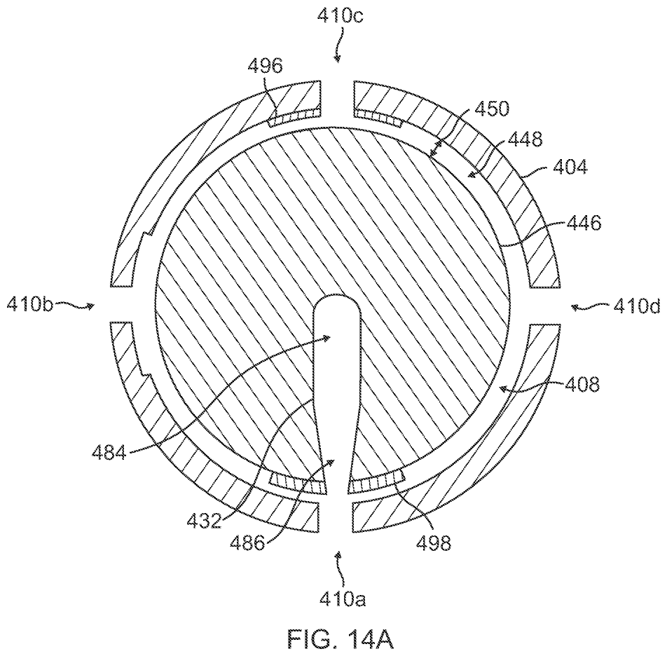

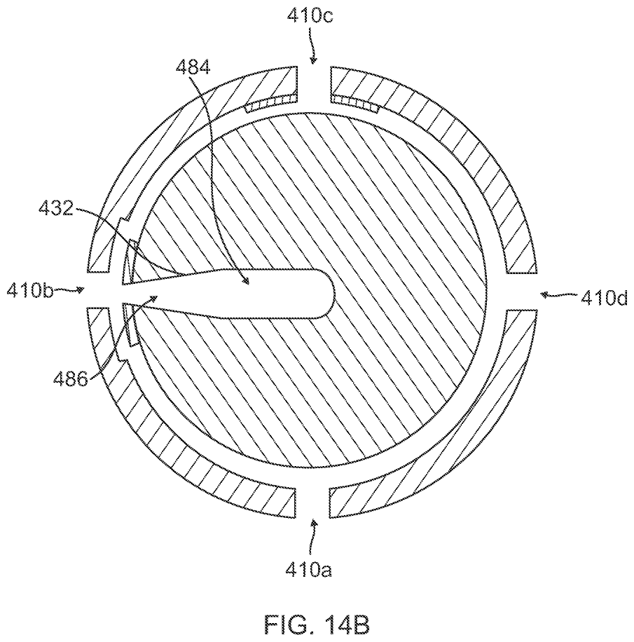

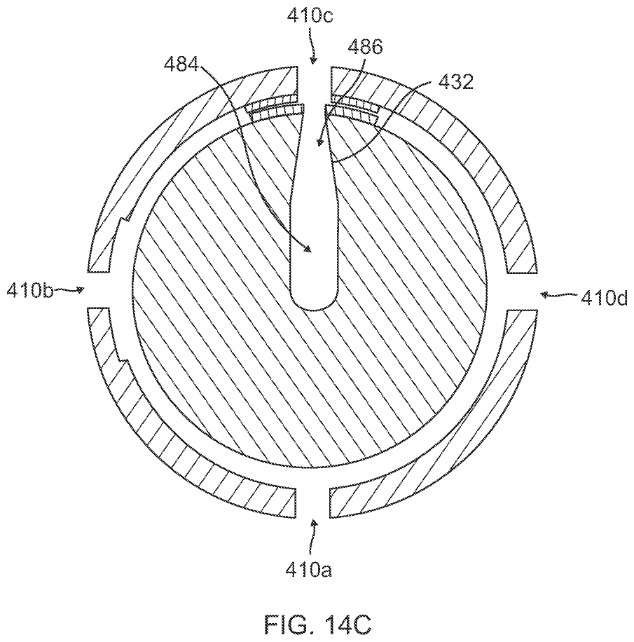

As reflected in, e.g., FIGS. 9 and 11, cavity 308 of stator body 304 has a cylindrical form defining an inner diameter of stator body 304 and portion 346 of rotor 326 also has a cylindrical form defining an outer diameter that is less than the inner diameter of the stator body. Therefore, there is a circular volume between portion 346 of rotor 326 and stator body 304, which may be considered a clearance. With reference to FIG. 14A, concerning an alternative design for a rotary fluidic distributor 400 that includes various similar structures to rotary fluidic distributor 300, including a cylindrical cavity/volute 408 and a cylindrical portion 446 of rotor 426, the volume between stator 404 and cylindrical portion 446 is indicated by reference numeral 448 and the clearance is indicated by reference numeral 450. The clearance may be less than about 0.01 inches, e.g., about 0.005 inches. Furthermore, as also reflected in FIG. 14A, the clearance need not be uniform. For example, the cross sections of either or both of portion 446 of rotor 426 and cavity 408 of stator body 404 need not be perfect circles. For example, proximate to the exit of a rotor conduit (e.g., 432), additional material 498 may be provided to reduce the clearance. Similarly, for example, proximate to a stator conduit (e.g., 410c), additional material 496 may be provided to reduce the clearance. Additionally, for example, less material may be provided about a stator conduit (e.g., 410b) to increase the clearance.

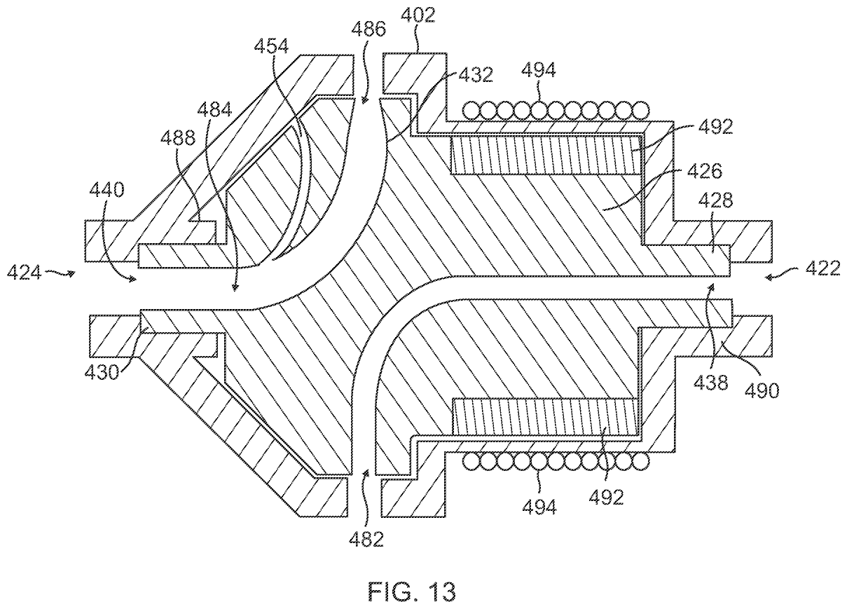

During operation of the rotary fluidic distributor (e.g., 300 or 400) the volume (e.g., 448) becomes filled with any fluid that is being flowed through the rotary fluidic distributor. Therefore, continuing with the example of rotary fluidic distributor 400, when a liquid is flowed through the rotor conduits (e.g., 432), it must enter volume 448, thus displacing some of liquid already contained in volume 448 into at least one of the stator conduits. When a given rotor conduit is aligned with one of the stator conduits, and particularly when they are precisely aligned, i.e., their central axes are coaxial, there is a straight path for liquid exiting that given rotor conduit to enter the aligned stator conduit. When the rotary fluidic distributor is in this condition, the aligned stator conduit receives a local maximum flow rate for that stator conduit. Of course, various variables and inputs affect the value of the local maximum flow rate, e.g., the diameter of the stator conduit, the diameter of the endoscope channel it is connected to, the overall number of rotor conduits and stator conduits, the number of rotor conduits that are aligned with stator conduits, and the flow rate of the liquid into the rotary fluidic distributor, which may itself be dynamic as controlled by a control system receiving feedback from a pressure sensor or a flow sensor. Conversely, for any given stator conduits that are not aligned with any rotor conduits, there is not a straight path from any rotor conduit into those stator conduits. In this condition, liquid exiting these rotor conduits enters volume 448, which displaces some of the liquid already in volume 448 to flow with flow rates less than the local maximum flow rates, e.g., local minimum flow rates, through these stator conduits. Further control or adjustment of flow rates into the stator conduits and ultimately to the endoscope channels connected thereto may be enabled by providing one or more bypass conduits 454 (FIG. 13) through the rotor. However provided, as used herein, the term “bypass conduit” is a rotor conduit that cannot ever be aligned with one or more, e.g., all, stator conduits.

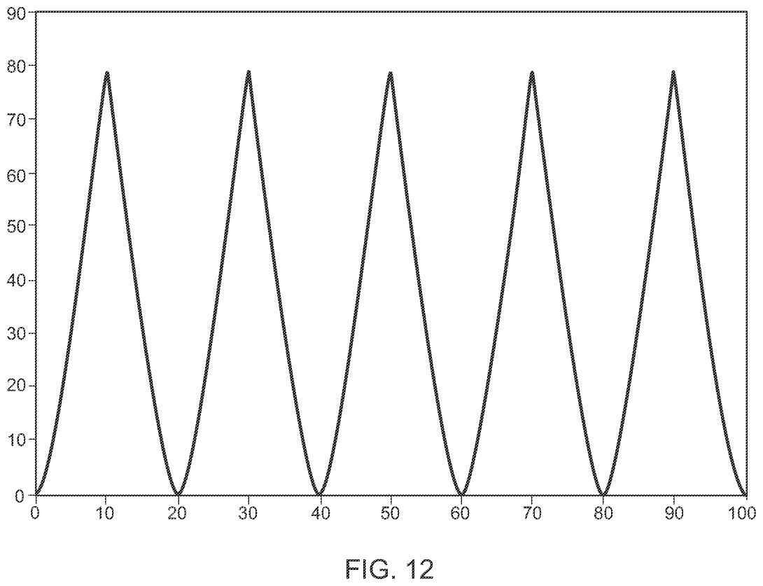

FIG. 12 graphically reflects a degree of alignment between a given stator conduit in either the first or second plane and the rotor conduits in that same plane, both of which have a radius of 5 millimeters, and hence an area of 78.5 mm². Where the alignment between any one of those rotor conduits and the stator conduit is maximized, a local maximum flow rate occurs in the stator conduit. Where the alignment between any one of those rotor conduits and the stator conduit is minimized, a local minimum flow rate occurs in the stator conduit.

FIG. 13 reflects a schematic representation of a side view of rotary fluidic distributor 400 to set forth an alternative manner of actuating a rotor of a rotary fluidic distributor. For simplicity, only a single set of rotor conduits and stator conduits are reflected. However, it should be understood that one or more sets of rotor conduits and stator conduits may be provided, similar to rotary fluidic distributor 300. In fact, unless otherwise specifically stated herein, rotary fluidic distributor 400 includes the same or similar structures and features as rotary fluidic distributor 300.

Rotary fluidic distributor 400 includes magnets for magnetically coupling rotor 426 and stator 402 to drive rotor 426. As shown in FIG. 13, rotor 426 comprises one or more permanent magnets 492 and stator 402 comprises one or more coiled wires 494, which may function as an electromagnet. Permanent magnets 492 may be provided inside material of rotor 426. Coiled wire 494 may be wrapped about stator 402 such that they the coil is disposed about the portion of rotor 426 containing magnets 492. Embedding magnets 492 in material of rotor 426 helps prevent liquids form contact them, which is a useful feature when caustic decontamination liquids, e.g., peracetic acid, are flowed through the rotary fluidic distributor. In this fashion, the functionality of a stepper motor, which is provided outside of stator 302 in rotary fluidic distributor 300, is integrated directly onto the stator and inside the rotor. Such does not require any modification to the rotor conduits or stator conduits between rotary fluidic distributors 300 and 400. Moreover, whether the actuator is a stepper motor as for distributor 300 or is a magnetic coupling as for distributor 400, the primary purpose of the actuator is to rotate the rotor in the stator. However, certain advantages are provided by the magnetic coupling. Some of these advantages include containing both first shaft 428 and second shaft 430 inside of stator 402 such that journal portions thereof may be constrained inside bearing portions of 488 and 490 of stator 402. Such enables connections of tubing for transporting decontaminants to both first shaft 428 and second shaft 430 because the second shaft need not be connected to a stepper motor located outside stator 402, as is the case for section shaft 330 and stepper motor 336. Accordingly, liquids may be flowed into rotor 426 through bore 438 of second shaft 428 and gases may be flowed into rotor 426 through bore 440 first shaft 430, or vice versa. Such optionally enables, as shown in FIG. 13, dedicated rotor conduits for liquid (e.g., 410) and dedicated rotor conduits for gas (e.g., 482). Furthermore, these connections do not require seals around rotating components, such as seals 332 and 334 around first shaft 328 and second shaft 330, because, in rotary fluidic distributor 400, first shaft 428 and second shaft 430 are disposed entirely inside stator 402. Although seal components may nonetheless be desirable wherever any tubing is connected to or through stator holes 422 and 424, any such seals may be stationary seals and not rotating seals.

FIGS. 13 and 14A-C reflect rotor conduit 432 as including a non-constricted portion 484 and a constricted portion or nozzle 486. Constricted portion 486 functions to increase the exit velocity of the liquid from the rotor conduit, which assists the liquid to cross volume 448 and enter any stator conduit that is aligned with it. The non-constricted portions of the rotor conduits (for rotary fluidic distributor 300 and 400) may have a diameter of between about 7 millimeters and about 13 millimeters, e.g., about 10 millimeters. The constricted portion may have a diameter that is between about 25% to about 75% smaller than the diameter of the non-constricted portion, e.g., about 50% smaller. The constricted portion may also be an orifice wherein the orifice may have a diameter that is between about 25% to about 75% smaller than the diameter of the non-constricted portion, e.g., about 50% smaller.

FIGS. 14A-C also reflect the motion of rotor portion 446 (and thus rotor 426) and three sequential instances in time. First, in FIG. 14A, rotor conduit 432 is aligned with stator conduit 410a, whereas no rotor conduit is aligned with any of stator conduits 410b-d. As such, the flow rate in stator conduit 410a is a local maximum flow rate while the flow rate in stator conduits 410b-d is less than a local maximum flow rate, e.g., a local minimum flow rate. The scenario reflected in 14B is similar to the scenario reflected in 14A, except that the rotor has rotated to align rotor conduit 432 with stator conduit 410b, meaning that the flow rate in stator conduit 410b is a local maximum flow rate while the other stator conduits do not have a local maximum flow rate. The scenario reflected in 14C is similar to the scenario reflected in 14A, except that the rotor has rotated to align rotor conduit 432 with stator conduit 410c, meaning that the flow rate in stator conduit 410c is a local maximum flow rate while the other stator conduits do not have a local maximum flow rate.

A rotary fluidic distributor may be integrated into an endoscope reprocessor, such as endoscope reprocessor 2. FIG. 16 reflects endoscope reprocessor 2 modified with rotary fluidic distributor 300, for example. Of course, rotary fluidic distributor 400 could be used as well. FIG. 16 is identical to FIG. 2 except that instead of the six pumps 32 shown as being individually connected along flush lines 30, only a single input line is connected to rotary fluidic distributor 300 (e.g., to first shaft 328 to feed the rotor conduits) and that only a single pump 32 is provided on that line upstream of rotary fluidic distributor 300. An ingress of each of the flush lines 30 is connected, one each, to each of the stator conduits. Although only six flush lines 30 are reflected in FIG. 16, two additional flush lines 30 may be provided for an endoscope that has eight channels to match the eight stator conduits of stator 302, or a rotary fluidic distributor with only six stator conduits may be provided.

When an endoscope having fewer channels than system 2 has flush lines is to be decontaminated in system 2, unused flush lines may be plugged or left unconnected. Alternatively, a rotary distributor having fewer stator conduits may be provided. Accordingly, the rotary fluidic distributor avoids the need to have a pump dedicated to each flush line. Moreover, it is also apparent that no valves are required between the single pump 32 and the endoscope to assist in pulsing the flow, directing the flow, or both.

An endoscope may be positioned in basin 14a and an egress of each of the flush lines 30 may be connected to the endoscope's channels. Where the rotary distributor is designed to provide different rates or a different pulse profile to endoscope channels having different diameters, care should be taken properly connect the endoscope's channels to the correct flush line. As noted above, some channels, e.g., the channels having smaller diameters may be pulsed between about thirty pulses per minute and about ninety pulses per minute, e.g., about sixty pulses per minute, whereas other channels, e.g., the channels having larger diameters may be pulsed between about two hundred pulses per minute and about four hundred pulses per minute, e.g., three hundred pulses per minute. Rotary fluidic distributor 300 may provide about three hundred pulses per minute to any endoscope channel connected to one of stator conduits 310a-b when rotor 326 is rotated with a frequency of about 0.25 hertz because there are twenty rotor conduits 332a-t that align with each of these stator conduits. Similarly, Rotary fluidic distributor 300 may provide about sixty pulses per minute to any endoscope channel connected to one of the stator conduits 312a-d when rotor 326 is rotated with a frequency of about 0.25 hertz because there are four rotor conduits 334 a-d that align with each of these stator conduits.

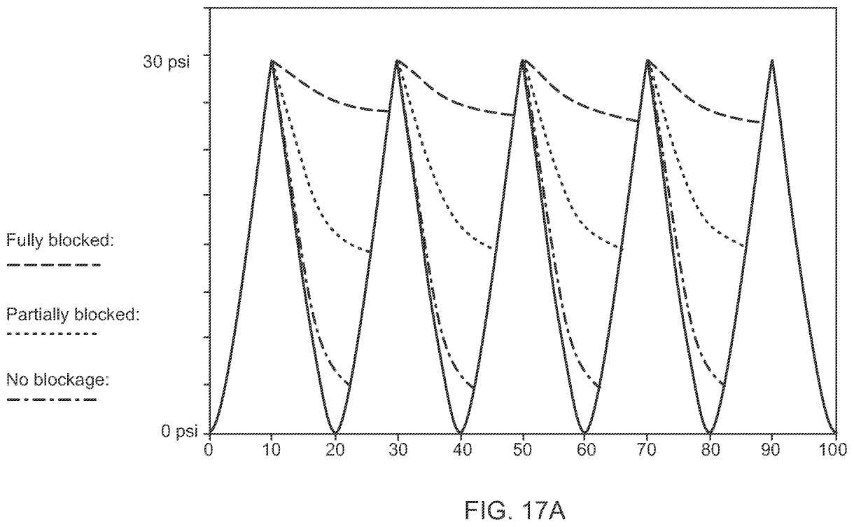

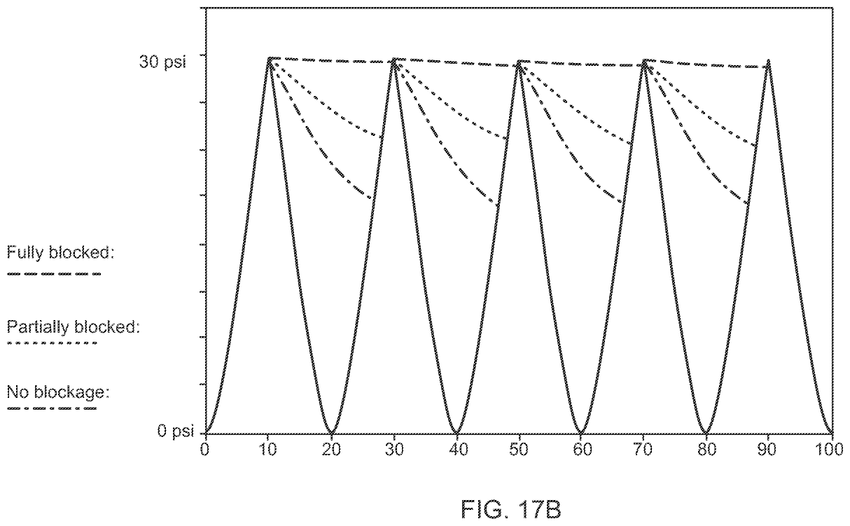

Applicant has also found that inclusion of a rotary fluidic distributor assists in detecting flow blockages downstream of the rotary fluidic distributor, including in channels of the endoscope. When such a blockage is present in an endoscope channel connected to a given stator conduit, a rapid pressure rise will occur as fluid is ejected into that stator conduit as a rotor conduit moves into alignment with that stator conduit. As the rotor conduit moves out of alignment with the stator conduit, i.e., away from maximum alignment (see FIG. 12 and related discussion, above), the pressure decays more slowly than it would if there were not blockage. As such pressure signatures can be determined for each channel of an endoscope corresponding to no blockage in the flow, various degrees of partially blocked flow, and fully blocked flow, such as is shown, for example, in FIGS. 17A and 17B. In these Figures, the solid line corresponds to the degree of alignment between a stator conduit and a rotor conduit, with the peaks corresponding to alignment of 100%. The dashed lines are the pressure-decay profiles. FIG. 17A reflects exemplary pressure-decay profiles for a large-lumen endoscope channel, i.e., a channel having a diameter of at least 1 mm, where the pump pressure is about equal to 30 psi. FIG. 17B reflects exemplary pressure-decay profiles for a small-lumen endoscope channel, i.e., a channel having a diameter of less than 1 mm, where the pump pressure is about equal to 30 psi.

By virtue of the embodiments illustrated and described herein, Applicant has devised a method and variations thereof for decontaminating an endoscope in an endoscope reprocessing system that includes a rotary fluidic distributor, such as rotary fluidic distributor 300 or 400. First, an endoscope (e.g., endoscope 200) may be placed into a basin (e.g., 14a) of a reprocessor 2. Second, flush lines 30 may be connected to the channels of this endoscope. Third, the reprocessor may be activated, which causes a decontaminant to flow first through the rotary fluidic distributor, then through the flush lines, and then through the channels of the endoscope. A first volume of the decontaminant may flow through the first set of rotor conduits (e.g., 332a-t) whereas a second volume may flow through the second set of rotor conduits (e.g., 334a-d). In accordance with the intended function of the rotary fluidic distributor, the rotor (e.g., 326) may be rotated while the decontaminant is flowing to pulse the flows of the first volume and the second volume. The pulsed flow may comprise between about thirty and about four hundred pulses per minute. Specifically, the pulsed flow may comprise between about thirty and about ninety pulses per minute, e.g., sixty pulses per minute, through the first flush line and between about two hundred and about four hundred pulses per minute, e.g., about three hundred pulses per minute through the second flush line. Furthermore, pulses of the pulsed flow may be provided through one of the flush lines out of phase with the pulses provided through another flush line. In other words, local maximum flow rates occur in one flush line at times when local maximum flow rates do not occur in another flush line.

The decontaminant may be flowed into the rotary fluidic distributor by pressures generated by a pump. For example, the pump may cause the decontaminant to flow at a rate of between about 100 milliliters per second and about 200 milliliters per second, e.g., about 160 milliliters per second.

Any of the examples or embodiments described herein may include various other features in addition to or in lieu of those described above. The teachings, expressions, embodiments, examples, etc., described herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but, in any order, as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

1. A rotary fluidic distributor, comprising:
a stator defining a stator longitudinal axis and a volute, and comprising a first set of stator conduits and a second set of stator conduits;
a rotor comprising a first set of rotor conduits and a second set of rotor conduits,
wherein the rotor is disposed in the volute of the stator such that a central first stator conduit axis of a first stator conduit of the first set of stator conduits and a central first rotor conduit axis of a first rotor conduit of the first set of rotor conduits are disposed in a first plane, and a central second stator conduit axis of a second stator conduit of the second set of stator conduits and a central second rotor conduit axis of a second rotor conduit of the second set of rotor conduits are disposed in a second plane,
wherein first plane is offset from the second plane along the stator longitudinal axis, and the central first stator conduit axis is not aligned with the central second stator conduit axis around the stator longitudinal axis.

2. The rotary fluidic distributor of claim 0, in which the central first and second stator conduit axises extend in a direction that is transverse to the stator longitudinal axis.

3. The rotary fluidic distributor of claim 0, in which the central first and second stator conduit axises extend in a direction that is perpendicular to the stator longitudinal axis.

4. The rotary fluidic distributor of claim 1, in which the rotor defines a rotor longitudinal axis that is coaxial with the stator longitudinal axis.

5. The rotary fluidic distributor of claim 1, in which the first stator conduit and the first rotor conduit are aligned.

6. The rotary fluidic distributor of claim 1, in which the first stator conduit and the first rotor conduit are not aligned.

7. The rotary fluidic distributor of claim 1, in which at least one rotor conduit of the first or second set of rotor conduits comprises a portion that is disposed radially through the rotor.

8. The rotary fluidic distributor of claim 1, in which at least one rotor conduit of the first or second set of rotor conduits is disposed nonradially through the rotor.

9. The rotary fluidic distributor of claim 1, in which the rotor further comprises a bypass conduit.

10-12. (canceled)

13. The rotary fluidic distributor of claim 1, in which the first set of rotor conduits are disposed above the second set of rotor conduits.

14. The rotary fluidic distributor of claim 1, in which the first set of rotor conduits comprises between fifteen to twenty-five rotor conduits.

15. The rotary fluidic distributor of claim 0, in which the first set of rotor conduits comprises twenty rotor conduits.

16. The rotary fluidic distributor of claim 1, in which the second set of rotor conduits comprises between three and five rotor conduits.

17. The rotary fluidic distributor of claim 0, in which the second set of rotor conduits comprises four rotor conduits.

18. The rotary fluidic distributor of claim 1, in which the rotor conduits of the first set of rotor conduits are equally spaced from each other.

19. The rotary fluidic distributor of claim 0, in which the rotor conduits of the second set of rotor conduits are equally spaced from each other.

20-21. (canceled)

22. The rotary fluidic distributor of claim 1, in which the first plane is disposed parallel to the second plane.

23-58. (canceled)

59. The rotary fluidic distributor of claim 1, in which at least one rotor conduit from among the first set of rotor conduits and the second set of rotor conduits includes a constricted portion and a non-constricted portion.

60. The rotary fluidic distributor of claim 0, in which the non-constricted portion has a diameter of between about 7 millimeters and about 13 millimeters.

61. The rotary fluidic distributor of claim 0, in which the non-constricted portion has a diameter of about 10 millimeters.

62. The rotary fluidic distributor of any of claims 0 to 0, in which the constricted portion has a diameter that is between about 25% to about 75% smaller than the diameter of the non-constricted portion.

63. The rotary fluidic distributor of claim 0, in which the constricted portion has a diameter that is about 50% smaller than the diameter of the non-constricted portion.

64-86. (canceled)

87. A rotary fluidic distributor, comprising:
a stator defining a stator longitudinal axis and a volute, and comprising a first set of stator conduits and a second set of stator conduits;
a rotor comprising a first set of between fifteen to twenty-five rotor conduits and a second set of between three and five rotor conduits,
wherein the rotor is and disposed in the volute of the stator such that a central first stator conduit axis of a first stator conduit of the first set of stator conduits and a central first rotor conduit axis of a first rotor conduit of the first set of rotor conduits are disposed in a first plane, and a central second stator conduit axis of a second stator conduit of the second set of stator conduits and a central second rotor conduit axis of a second rotor conduit of the second set of rotor conduits are disposed in a second plane.
